(19) United States
(12) Patent Application Publication
(10) Pub. No.: US 2025/0367403 A1
(43) Pub. Date: Dec. 4, 2025

(54) Devices and Methods for Forming Eyelets in Urinary Catheters

(71) Applicant: Hollister Incorporated, Libertyville, IL (US)

(72) Inventor: Joseph Fahey, Newport (IE)

(21) Appl. No.: 18/874,621

(22) PCT Filed: Jun. 9, 2023
(86) PCT No.: PCT/US2023/068220

§ 371 Date: Dec. 13, 2024

Related U.S. Application Data
(60) Provisional application No. 63/352,095, filed on Jun. 14, 2022.

(51) Int. Cl. A61M 25/00

(52) U.S. Cl. CPC A61M 25/0015; A61M 25/0017

(57) ABSTRACT

Methods and systems for forming eyelets in urinary catheters are provided.

### CROSS-REFERENCE TO RELATED APPLICATIONS

This application claims the benefit of U.S. Provisional Application Ser. No. 63/352,095, filed Jun. 14, 2022, the disclosure of which is hereby incorporated by reference in its entirety.

### TECHNICAL FIELD

The present disclosure generally relates to devices and methods for creating holes in catheters, and more specifically, to systems and methods for creating at least one eyelet in a urinary catheter.

### BACKGROUND

Medical tubing, such as urinary catheters, are used for a wide range of medical and surgical applications, e.g., catheterization for the drainage of urine from the bladder. Catheters are generally polymer tubes that have an insertion end that is advanced through a lumen of the patient to a desired location within the body. The insertion end typically includes a hole or eyelet therein to allow substances to pass into or out of the lumen of the catheter shaft.

Urinary catheter tubes are used to drain urine from the bladder. The catheter tube includes a lumen and a hole/eyelet in the insertion end of the catheter tube. The hole/eyelet is in fluid communication with the lumen of the catheter tube so as to allow urine to drain from the bladder and through the lumen of the tube. In use, the insertion end of the catheter tube is advanced through the urethra of the user and into the bladder. Once in the bladder, urine drains from the bladder through the hole/eyelet in the insertion end and into the lumen of the catheter. The urine is discharged through the distal end of the catheter tube.

There are various considerations for forming a hole/eyelet in urinary catheters. Such considerations may include costs, speed of cutting, efficiency, punched/cut out parts remaining in the shaft, etc.

Therefore, there remains a need for improved systems and methods for forming eyelets in urinary catheters.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a method of forming at least one eyelet in a catheter shaft. The method includes cooling at least a portion of the shaft and punching at least one eyelet in the cooled portion of the catheter shaft.

In another aspect, a system for forming at least one eyelet in a catheter shaft. The system includes a stencil for placing over a catheter shaft with at least one opening, wherein the at least one opening corresponds to an eyelet for the catheter, a mandrel for inserting into a catheter shaft, a device for cooling at least a portion of the catheter shaft, and a machining device for punching eyelet holes into the catheter shaft. Optionally, the mandrel and/or the stencil may be at a selected temperature that cools at least a portion of the catheter shaft. Alternatively, or in addition to the mandrel and/or stencil cooling a portion of the catheter shaft, the system may include a separate cooling device or component. Such a cooling device may be a device that delivers cool gas (for example, air) or a cooling liquid onto the portion of the catheter shaft.

### BRIEF DESCRIPTION OF FIGURES

FIG. 1 is a perspective view of one example of a catheter having an eyelet therein;

FIG. 2 is a perspective view of the catheter, a stencil and a mandrel of an exemplary embodiment;

FIG. 3 is a perspective view of a catheter and a stencil according to an exemplary embodiment;

FIG. 4 is a schematic cross-sectional view of a catheter inserted into a machining device with a cooling device and mandrel;

FIG. 5 is a schematic cross-sectional view of a catheter inserted into a machining device with a mandrel and stencil according to an exemplary embodiment; and

FIG. 6 is a schematic cross-sectional view of a catheter inserted into a machining device with a mandrel and stencil according to an exemplary embodiment.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

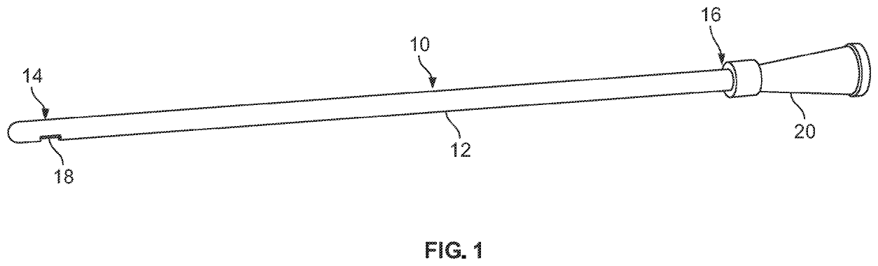

The present disclosure is directed to devices and methods for creating a hole in the wall of a medical tubing. Turning to FIG. 1, there is shown a urinary catheter 10. Although aspects of the present disclosure are described relative to a urinary catheter. the devices and methods disclosed herein may be used to make a hole in any medical tubing, such vascular catheters, exploratory devices, etc. The medical tubing may be made from a flexible or elastic material, such as a polymer. The polymer may be a thermoplastic polymer, for example, polyvinyl chloride, EVA, PEBAX, SEBS, etc.

The urinary catheter 10 includes a catheter shaft 12 (which is a tubing) having a proximal end portion 14 and a distal end portion 16. At least one eyelet 18 extends through the wall of the catheter shaft 12 and is in communication with a lumen (not shown) of the catheter shaft. In the illustrated embodiment, the hole 18 is in the proximal end portion 14 of the catheter shaft 12. A drainage member 20, such as a funnel or connector, may be attached to the distal end portion 16 of the catheter shaft 12. The drainage member 20 is in communication with the lumen of the catheter shaft 12 for discharge of urine from the shaft 12.

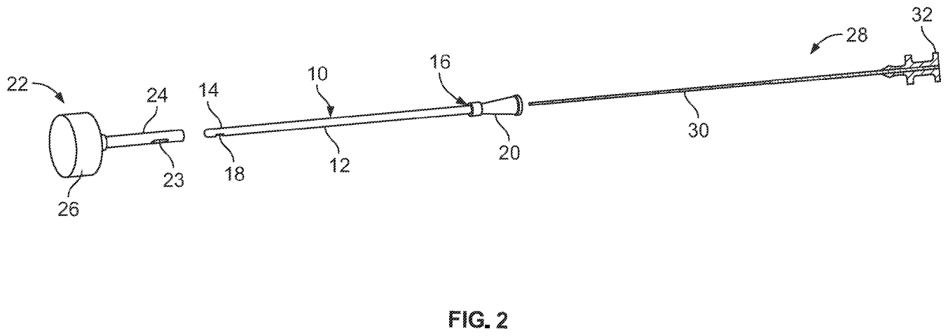

FIG. 2 shows the urinary catheter 10 in combination with a stencil 22 and a mandrel 28, which may be used in combination or separately. As shown in FIG. 2, the stencil 22 may be inserted or applied over the proximal end portion 14 of the urinary catheter shaft 12 and the mandrel 28 may be inserted or applied into the lumen of the catheter shaft 12 through the distal end portion 18 of the catheter shaft 12. The stencil 22 includes a base 26 and a hollow barrel 24 extending therefrom. The base 26 may be generally cylindrically shaped or it may have any other suitable shape. The base 26 may be configured for connecting the stencil 22 to an advancing machine or device. The barrel 24 includes an end opening for receiving the catheter shaft 12 and at least one opening 23 in the side wall which corresponds to the eyelet 18 on the catheter 10. The mandrel 28 includes a rod 30 that is configured for insertion into the lumen of the catheter shaft 12 and a connector 32 for attaching to an advancing machine or device.

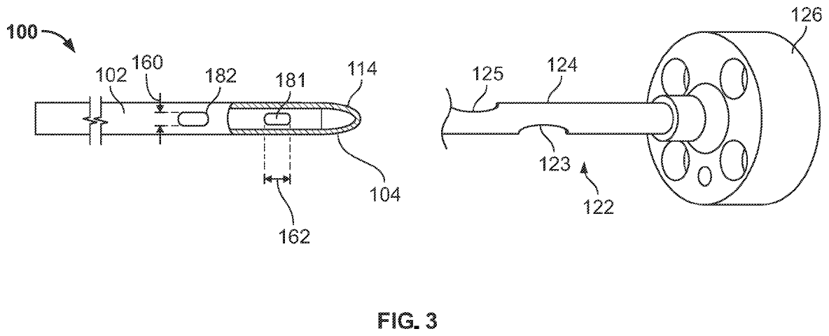

FIG. 3 shows another urinary catheter 100 in combination with a stencil 122. Although not shown in the figure, the mandrel, or a similar mandrel, of FIG. 2 may also be used in combination with the catheter 100 and stencil 122 of FIG. 3. The stencil 122 includes a barrel 124 with two openings 123 and 125. After holes are created in the catheter 100, the catheter 100 will includes two eyelets 181 and 182. The first eyelet 181 will be on the first or a bottom side 104 of the catheter and the second eyelet 182 will be on the second or a top side 102 of the catheter 100. The first opening 123 of the barrel 124 of the stencil 122 corresponds to the first eyelet 181/location thereof and the second opening 125 of the barrel 124 corresponds to the second catheter eyelet 182/location thereof when the barrel 124 of the stencil 122 is placed over the catheter shaft and aligned.

The barrel 124 of the stencil 122 is shaped similarly to the catheter shaft. The barrel 124 of the stencil 122 can be a hollow tube or have a hollow tube portion with an open end for sliding over the catheter shaft. The barrel 124 has a sufficient inner diameter to be placed over the urinary catheter, i.e., the inner diameter is slightly larger than the outer diameter of the urinary catheter shaft. For example, the inner diameter of stencil's barrel may be in the range of from about 2.2 mm up to about 6.2 mm (CH06-CH18) depending on the Charriere (Diameter) of the catheter in question and the tolerance of the tube diameter. Near the end of the stencil is at least one opening corresponding to the desired eyelet shape that will be formed in the urinary catheter shaft. In one alternative there are at least two openings. In one example, the two openings in the barrel correspond to a desired two eyelet pattern in the urinary catheter. The openings may be arranged on the same side of the stencil or on opposite sides of the stencil. In one embodiment, the openings are arranged on opposite sides of the stencil (shown in FIG. 3) which is more typical.

The openings, which will correspond to the shape of the catheter eyelets, can be any desired shape or size and customized based on the application. The openings may have rounded edges and may form circles or ovals. Typically, the openings may have a racetrack or oval/elliptical type shape. This may depend on the structural integrity of the tube and so other variants could be possible. The openings can be a range of sizes, depending on the size of the catheter and application. The length of the opening 181, which is shown in FIG. 3 as 162, may be in the range of about 2.0 mm to about 5.0 mm. The width, which is shown in FIG. 3 as 160, may be in the range of about 0.5 to about 2.0 mm.

The stencils can be composed of metal or metallic material. In one embodiment, the stencil is composed of stainless steel. The stencil can also be composed of a polymeric material, such as polyurethane, or a ceramic material. It could also be a hybrid material as in Metal/Polymeric, where the metal gives the rigidity and the polymeric allows for the punching surface. It could also include a balloon/bladder type system that could be a part of or attached to stencil and expand in the region of eyelet both for support and as a mechanism of cooling.

The stencil may be advanced by any known advancing system. This can be done mechanically, pneumatically, hydraulically, or ultrasonically. Any advancing system can be used that can advance and retract the stencil from the urinary catheter. By way of example, the advancing system may be electromechanically actuatable, utilizing electrical, pneumatic, or hydraulic fluid pressure energy. This can also be done ultrasonically. In one embodiment, the advancing system may comprise a screw, an axle, and a connection member.

The advancing system can be flexible or rigid and can be a chain. belt or cable or a rod or shaft. In yet another exemplary embodiment, the advancing system can comprise a cam and a motor which is driven in forward and reverse directions to rotate the cam in opposite directions, thereby moving the stencil in opposite directions. The advancing system may alternatively (or additionally) include a magnetic linear motor. In another embodiment, the advancing system may include a telescoping member.

Referring to FIG. 2, the stencil 22 may be advanced over the catheter shaft 12 from the proximal end portion 14 of the catheter shaft, as shown in FIG. 2. The stencil may also be advanced over the catheter shaft 12 from the distal end portion 16 of the catheter shaft 12. The stencil and mandrel may be advanced from opposite sides of the catheter, i.e., if the stencil is advanced over the proximal end of the catheter, the mandrel is advanced through the distal end of the catheter and vice versa. In another embodiment, the stencil and mandrel are formed as part of the same tool and advanced from the same direction. The mandrel is a tube within the stencil tube and spaced far enough apart that the catheter can fit in between.

The mandrel 30 is also similarly shaped to the urinary catheter shaft 12. The mandrel 30 may be a rod having an outer diameter that is slightly smaller than the inner diameter of the urinary catheter shaft 12 so that the mandrel 30 may be inserted into the catheter shaft 12 and provide support or structural integrity for the catheter shaft while punching the eyelet(s). Accordingly, the mandrel 30 may be continuous, or without any interruptions or openings. The mandrel 30 can be solid or an open hollow rod.

The mandrel 30 can be composed of metal or metallic material. In one embodiment, the mandrel 30 is composed of stainless steel. The mandrel 30 can also be composed of a polymeric material, such as polyurethane, or a ceramic material. It could also be a hybrid material as in Metal/Polymeric, where the metal gives the rigidity and the polymeric allows for the punching surface, or instead metal/ceramic etc.

The mandrel 30 may be advanced into the lumen of the catheter shaft 12 from the distal end portion 16 of the catheter shaft, as shown in FIG. 2. The mandrel 30 may also be advanced into the lumen of the catheter shaft 12 from the proximal end portion 14 of the catheter shaft 12 before the catheter is tipped. The stencil and mandrel may be advanced from opposite sides of the catheter, i.e., if the stencil is advanced over the proximal end of the catheter, the mandrel is advanced through the distal end of the catheter and vice versa. In another embodiment, the stencil and mandrel are formed as part of the same tool and advanced from the same direction. The mandrel is a tube within the stencil tube and spaced far enough apart that the catheter can fit in between.

The mandrel 30 material may be robust enough to withstand the punching tool for at least 100,000 cycles (one cycle is punching two eyelets). However, in at least one embodiment, the mandrel does not need to come in contact with the rod of the mandrel and purely acts as a support for the punching of the eyelet out of the catheter shaft.

As with the stencil, the mandrel may be advanced by any known advancing system. This can be done mechanically, pneumatically, or hydraulically. Any advancing system can be used that can advance and retract the mandrel from the urinary catheter. By way of example, the advancing system may be electromechanically actuatable, utilizing electrical, pneumatic, or hydraulic fluid pressure energy. This can also be done ultrasonically. In one embodiment, the advancing system may comprise a screw, an axle, and a connection member. The advancing system can be flexible or rigid and can be a chain, belt or cable or a rod or shaft. In yet another exemplary embodiment, the advancing system can comprise a cam and a motor which is driven in forward and reverse directions to rotate the cam in opposite directions, thereby moving the mandrel in opposite directions. The advancing system may alternatively (or additionally) include a magnetic linear motor. In another embodiment, the advancing system may include a telescoping member.

Prior to the punching of the eyelets, the catheter is cooled to a particular temperature range in order to aid the punching process. The cooling temperature is dependent on the type of material. In one alternative, the at least a portion of the catheter shaft 12 is cooled to a temperature below room temperature (below 23° C.). In another alternative, at least a portion of the catheter shaft 12 is cooled to a temperature below 6° C. In yet another alternative, at least a portion of the catheter shaft 12 is brought to a temperature of between about −40° C. and about 5° C. The eyelet is created in the portion of the catheter that has been cooled and while it is still cold. In other embodiments, the catheter shaft is made from polyvinyl chloride (PVC), and at least a portion of the catheter shaft is cooled to from about −30° C. to about 5° C. In one alternative, at least a portion of the catheter shaft comprised of elastomer (TPE) is cooled to from about −19° C. to about 4° C. In another alternative, at least a portion of the catheter shaft comprised of thermoplastic olefin (TPO) is cooled to from about −40° C. to about 4° C.

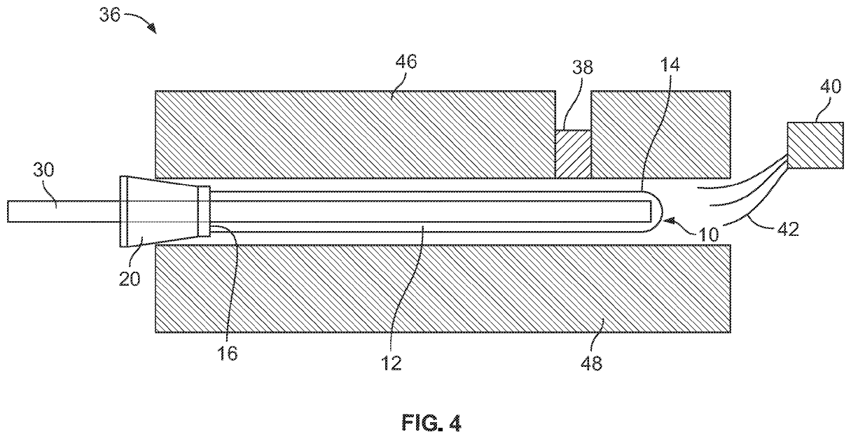

The cooling can happen via several mechanisms. As shown in FIG. 4, the catheter may be cooled by a separate cooling device 40. The device 40, as shown in the figure, may be adjacent to the proximal end portion 14 of the catheter shaft 12 so as to efficiently cool the proximal end portion 14 of the catheter shaft 12. The cooling device 40 may be alternately situated, such as within the machining device, and may be connected or used in conjunction with the any or all of the punches. The cooling device 40 may use any type of cooling fluid. For instance, the device may utilize air cooling, water or liquid cooling, liquid dispense/evaporation cooling, and/or a combination of two or more types or fluids for cooling.

The cooling may also be done by one or both of the stencil and mandrel. One or both of the mandrel and stencil may have hollow walls so that they may be cooled via cooling fluid or compressed air and in turn cool the catheter shaft 12. This cooling may take place while the mandrel and/or stencil is in contact with the catheter, or it may be done before being advanced in or placed on the catheter. In one embodiment, the mandrel may be inserted into the catheter shaft of the catheter and then expanded via air cooling or liquid and come into contact with the walls of the catheter shaft in order to cool it. The stencil and/or the mandrel may be at a selected cooling temperature. For example, the stencil and/or mandrel may be between about −50° C. and about 5° C. In one alternative, the stencil and/or mandrel may be cooled below 0° C. In another alternative, the stencil and/or mandrel may be cooled below 5° C.

It may be beneficial to monitor the temperature of the shaft in order to verify that catheter shaft is at the correct temperature before punching. This could be done with a closed-loop thermocouple embedded in the walls of the mandrel and/or stencil, so that temperature feedback is confirmed prior to commencing the punching process. It may be possible also to use thermal imaging to give confirmation that the shaft is at the correct temperature before its punched. Any known temperature monitoring device can be used. The temperature of the cooling fluid may be also regulated so that it is kept in a specific range that will cool the catheter shaft efficiently and effectively.

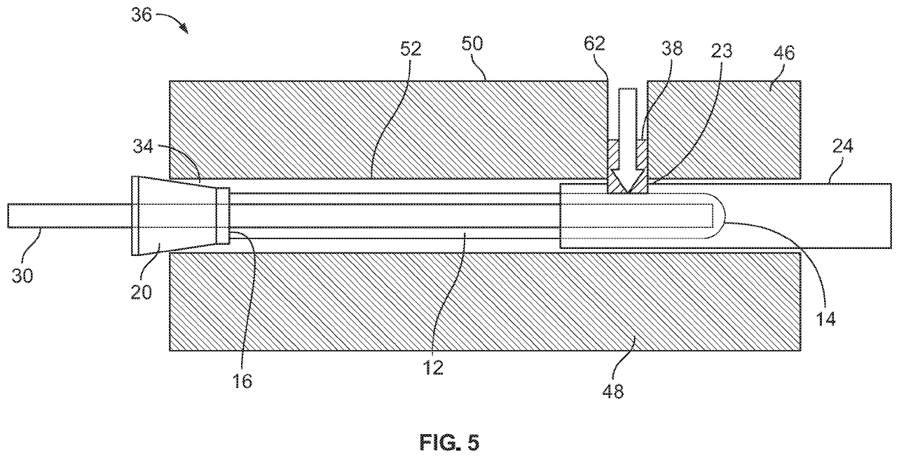

FIG. 5 illustrates one embodiment of a machining device for forming an eyelet in a catheter shaft 12. The machining/Punching device may be used, for example, to form at least one eyelet 18 in the catheter 12 shown in FIG. 1. The machining/punching device 36 includes a pair of saddles/supports, bottom saddle or member 48 and top saddle/support or member 46. The terms “bottom” and “top” are arbitrarily used herein for convenience in describing that the saddles border the channel for the urinary catheter. It will be understood that these terms are not being used to limit the saddles/supports to “top” and “bottom” positions. These may be arranged vertically and have first and second sides.

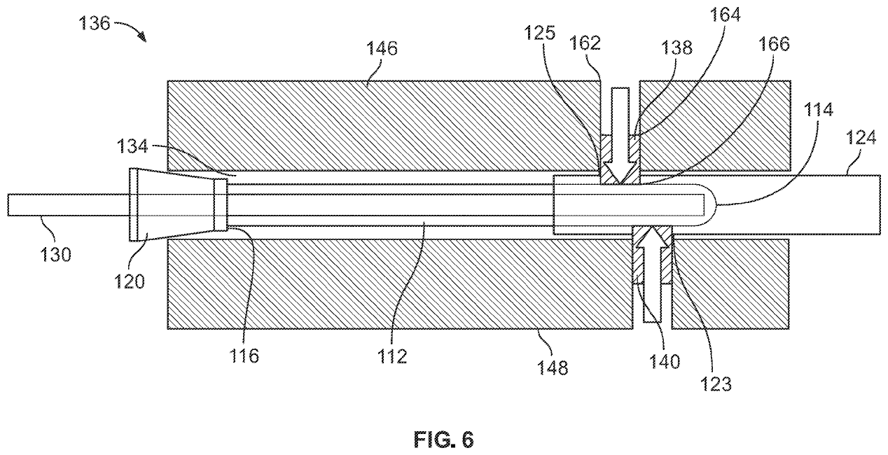

The machining device 36 also may include at least one punch 38. The top saddle 46 includes a punch receiving aperture 62 extending therethrough from the top surface 50 of the saddle to the bottom surface 52 of the saddle. The aperture 62 may be any shape, and may have a shape that corresponds, is commensurate and/or otherwise allows or guides the top punch 38 therethrough. In some embodiments, the system includes at least two punches (FIG. 6). In the illustrated embodiment of FIG. 5, the punch is in the top saddle. In alternate embodiments the punch can be in the bottom saddle. In some embodiments, at least two punches are present. In these embodiments the at least two punches may both be in the same saddle, or in different saddles. In one such embodiment, as shown in FIG. 6, one punch 138 is in the top saddle and a second or bottom punch 140 is in the bottom saddle. After a portion of the catheter shaft 12 is cooled, the punch 38, 138 is advanced or moved through apertures in the saddle and an opening 23, 123 in the stencil 24, 124 to cut a portion of the tubing, thereby forming the eyelet. The punch 38, 138 may be inserted into the apertures or may reside within the apertures and be actuated to move or advance within the apertures. Furthermore, the cooling device may cool the catheter shaft through the apertures of the saddles. For example, after the shaft 12 is placed within the saddles, cold gas or liquid may be delivered through the aperture(s) of the saddle. In another alternative, a cooling structure, such as a cooling rod or even the punch itself could come into contact with outer surface the shaft, thereby cooling the shaft before punching.

In the embodiments of FIG. 5 and FIG. 6, the punches 38, 138, and 140 are similarly configured and have a shape that is commensurate with the respective aperture. However, in an alternative, the punches may be differently configured.

In the illustrated embodiments, each punch includes a shaft having a distal end 164 and a proximal end 166. The distal end 164 may be operatively connected to a device that actuates the punch or punches so that they move within respective apertures of the top and bottom saddle. The proximal end 166 of the punch is a cutting end that includes a cutting element that is configured to cut a hole in the shaft. The cutting element may be, for example, a blade. The blades are used to cut an eyelet in the catheter.

The distal end 164 of the punch 138 is connected to an advancing system for moving the punch. The punching of the machining device can be done mechanically, pneumatically, hydraulically, or by a cam type system. The punching can also be ultrasonic. By way of example, the punch may be electromechanically actuatable, utilizing electrical, pneumatic, or hydraulic fluid pressure energy.

The machining device 36, 136 includes a channel 34, 134 therein for receiving and holding the urinary catheter. The channel 34, 134 may be defined by at least one wall of the bottom saddle 48, 148 and at least one wall of the top saddle 46, 146. The first and second ends of the channel may be open. This allows for the catheter to move in and out and also for the cooling device, mandrel, and stencil to move in and out freely. The channel may be the length of the catheter or only a portion of the length of the catheter.

The punched portion of the catheter can be removed by any applicable process. It can be removed by vacuum. It can also be directly pumped into the machining device. Another possible method is to mechanically eject the slug. It may be that if its pressurized air/liquid cooling that removes the slug.

Using the system described above, at least one eyelet may be punched in a catheter shaft. The method includes cooling at least a portion of the catheter shaft and punching at least one eyelet into the catheter shaft. The portion of the catheter shaft is preferably the proximal portion of the catheter shaft, but any portion of the catheter shaft where an eyelet is desired may be cooled. In one embodiment, the entire catheter is cooled.

The method may also include inserting a mandrel into an end of the catheter shaft. The method may also include placing a stencil over at least a portion of the catheter shaft. Both inserting the mandrel and placing the stencil ·10- may be done before the punching of at least one eyelet.

The cooling step of the method can be accomplished by applying a fluid, such as air or gas to at least a portion of the catheter shaft by a separate device. As mentioned above, the mandrel and/or stencil may also be cooled in order to cool at least a portion of the catheter shaft. The mandrel may be cooled before inserting into the catheter shaft or while inserted in the catheter shaft. The stencil may also be cooled before placing over the catheter shaft or while placed over the catheter shaft.

The punching of at least one eyelet into the catheter shaft may include punching at least two eyelets into a catheter shaft. The punches may be done simultaneously or consecutively. If the punches are done simultaneously a stencil may be used with two openings corresponding to the pattern of eyelets desired. The stencil is placed over the catheter shaft and two punches from the machining device are advanced into the catheter shaft, forming the eyelets. If done consecutively, a stencil with a single opening may be used and a machining device with a single punch may also be used. In this case, the stencil may be advanced along the catheter shaft so that the opening aligns with a first desired eyelet location of the catheter shaft and the eyelet punched. Afterward, the catheter may be moved so that the stencil and punch align with a second desired eyelet location of the catheter shaft. If the shaft 12 is repositioned it may be moved axially and/or rotated about its longitudinal axis to create subsequent holes.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A method of forming at least one eyelet in a catheter shaft with a proximal end portion and a distal end portion, comprising:
cooling at least a portion of the catheter shaft; and
punching at least one eyelet in the at least a portion of the catheter shaft.

2. The method of claim 1, further comprising inserting a mandrel into a lumen of the catheter shaft.

3. The method of any one of claim 1, further comprising placing a stencil over at least a portion of the catheter shaft, the stencil having openings corresponding to a location of the at least one eyelet in the catheter shaft.

4. The method of claim 3, wherein the placing of the stencil is done before punching at least one eyelet in the catheter shaft.

5. The method of claim 3, wherein the placing of the stencil is before cooling at least a portion of the catheter shaft and the stencil is at a selected cooling temperature, the cooling at least a portion of the catheter shaft comprising cooling the catheter shaft by contact with the stencil.

6. The method of claim 2, wherein the inserting of the mandrel is done before punching at least one eyelet in the catheter shaft.

7. The method of claim 6, wherein cooling at least a portion of the catheter shaft and the mandrel is at a selected cooling temperature, the cooling at least a portion of the catheter shaft comprises cooling the catheter shaft by contact with the mandrel.

8. The method of claim 1, wherein the cooling at least a portion of the catheter shaft includes applying air or gas to at least a portion of the catheter shaft.

9. The method of claim 1 wherein the portion of the at least a portion of the catheter shaft comprises a proximal portion of the catheter shaft.

10. The method of claim 2, wherein the mandrel is inserted from a distal end of the catheter shaft.

11. The method of claim 3, wherein the stencil is placed over the proximal end portion of the catheter shaft.

12. The method of claim 1, wherein the punching includes punching at least two eyelets.

13. The method of claim 12, wherein the punching of at least two eyelets is done simultaneously.

14. The method of claim 12, wherein the punch of at least two eyelets is done consecutively.

15. The method of claim 1, wherein cooling of the at least a portion of the catheter shaft comprises cooling the at least a portion of the catheter shaft to a temperature of between about −40° C. to about 5° C.

16. The method of claim 1, wherein the catheter shaft comprises PVC and wherein cooling of the at least a portion of the catheter shaft comprises cooling the at least a portion of the catheter shaft to a temperature of between −30° C. to about 5° C.

17. The method of claim 1, wherein the catheter shaft comprises TPE and wherein cooling of the at least a portion of the catheter shaft comprises cooling the at least a portion of the catheter shaft to a temperature of between −19° C. to about 4° C.

18. The method of claim 1, wherein the catheter shaft comprises TPO and wherein cooling of the at least a portion of the catheter shaft comprises cooling the at least a portion of the catheter shaft to a temperature of between −40° C. to about 4° C.

19. A system for forming at least one eyelet in a catheter including a catheter shaft with a proximal end and a distal end, comprising:
a stencil for placing over a catheter shaft with at least one opening, wherein the at least one opening corresponds to at least one eyelet of the catheter shaft;
a mandrel for inserting into a catheter shaft;
a device for cooling at least a portion of the catheter shaft; and
a machining device for punching eyelet holes into the catheter shaft with a channel for receiving at least a portion of the catheter.

20. The system of claim 19, wherein the device for cooling is a separate cooling device.

21.-26 (canceled)
